(19) United States
(12) Patent Application Publication
(10) Pub. No.: US 2015/0238142 A1
(43) Pub. Date: Aug. 27, 2015

(54) Mouth guard with Visual Concussion Indicator System

(71) Applicant: Vancho Djordjevski, Chesterfield, MI (US)

(72) Inventor: Vancho Djordjevski, Chesterfield, MI (US)

(21) Appl. No.: 14/632,546

(22) Filed: Feb. 26, 2015

Related U.S. Application Data
(60) Provisional application No. 61/945,336, filed on Feb. 27, 2014.

(51) Int. Cl. A61B 5/00; A63B 71/08; A61B 19/00

(52) U.S. Cl. CPC A61B 5/682; A61B 19/46; A61B 5/742; A63B 71/085; A61B 2019/465; A61B 2503/10; A61B 2560/0462; A61B 2562/043; A63B 2071/086; A61B 2562/0247; A61B 2562/164

(57) ABSTRACT

An apparatus that can be used to detect concussion causing forces in an athlete contains a mouth guard, a plurality of force-sensitive visual indicators, and a plurality of transparent covers. The plurality of force-sensitive visual indicators is used to indicate the occurrence of a significant impact. When the apparatus is utilized, the athlete places the upper set of teeth in a teeth-receiving channel of the mouth guard. When the athlete experiences a significant impact, the plurality of force-sensitive visual indicators is activated. Since the plurality of force-sensitive visual indicators is enclosed within a U-shaped body of the mouth guard, the athlete or another observer can get an understanding of the severity of the impact by examining the plurality of force-sensitive visual indicators. In particular, the plurality of force-sensitive visual indicators is examined through the plurality of transparent covers.

The current application claims a priority to the U.S. Provisional Patent application Ser. No. 61/945,336 filed on Feb. 27, 2014.

### FIELD OF THE INVENTION

The present invention relates generally to protective equipment. More specifically, the present invention is a mouth guard that provides a visual indication of the probability that the wearer has sustained a concussion.

### BACKGROUND OF THE INVENTION

Concussions are one of the most common types of brain injuries. Concussions are particularly common in athletes of all levels who participate in sports where the likelihood of head impacts is common. Much of the danger surrounding concussions stems from the fact that many head injuries are regularly unreported and/or undiagnosed. This is due to the fact that the damage resulting from a head injury is often underestimated due to the lack of immediate noticeable symptoms. The majority of symptoms of minor concussions are typically resolved in 7-10 days through rest and a gradual return to normal activities. However, in certain cases, complications may arise, resulting in permanent thinking, sleeping, and balancing problems. In rare cases, a life-threatening condition such as the formation of a blood clot on the brain may occur. Although protective headgear is generally sufficient in preventing serious traumatic injuries to the brain, the headgear does not provide any indicator of whether or not the wearer has sustained a concussion. The present invention seeks to address the aforementioned issue while providing users with a practical and convenient solution.

The present invention is a mouth guard that features an indicator system that provides a visual cue of the probability that the wearer has sustained a concussion. The present invention provides an indicator system for concussions while providing the full functionality of a mouth guard. In its preferred embodiment, the present invention comprises a mouth guard that houses a plurality of impact indicators. Three impact indicators are embedded within hollow housing spaces located on the front, left, and right sides of the mouth guard. The impact indicators are filled with a chemical dye that changes color from white to red upon experiencing an impact greater than or equal to 37.5 g of force. A common indicator threshold for concussions is approximately 50 g of force with an impact of 100 g of force very likely resulting in a concussion. The housing spaces are covered by translucent material. As a result, the color of the impact indicators is easy visible.

The object of the present invention is to provide users with an unobtrusive device that serves the dual purpose of a mouth guard and a concussion indicator. While the present invention is not capable of guaranteeing that the wearer has sustained a concussion, the changing colors of the embedded impact indicators notify users that a precautionary medical examination is prudent following an impact.

### BRIEF DESCRIPTION OF THE DRAWINGS

FIG. 1 is a perspective view of the present invention.

FIG. 2 is a front view of the present invention.

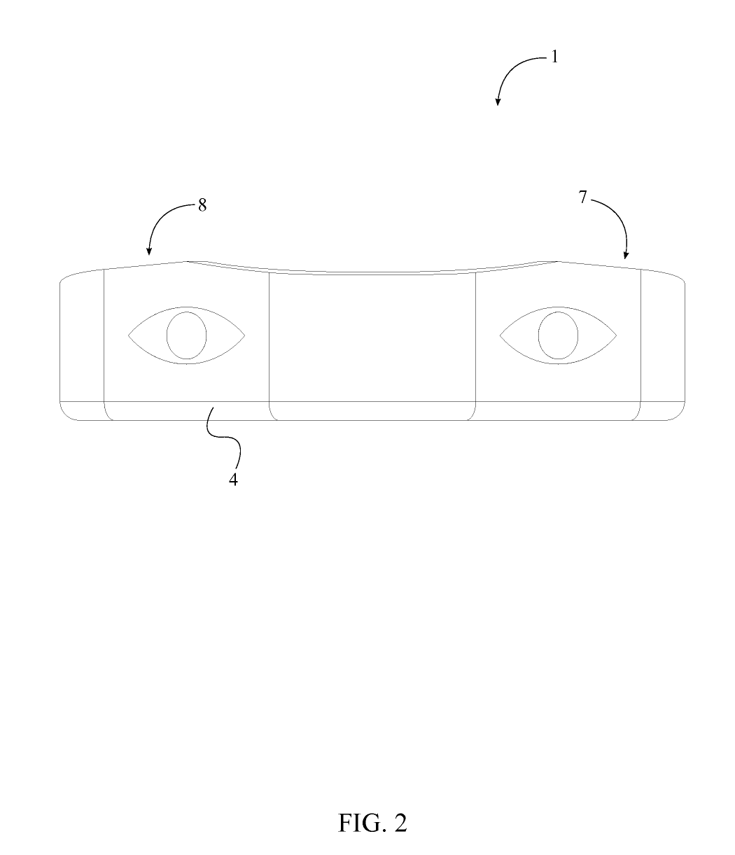

FIG. 3 is a rear view of the present invention, wherein the plurality of force-sensitive visual indicators is in the first configuration of the present invention.

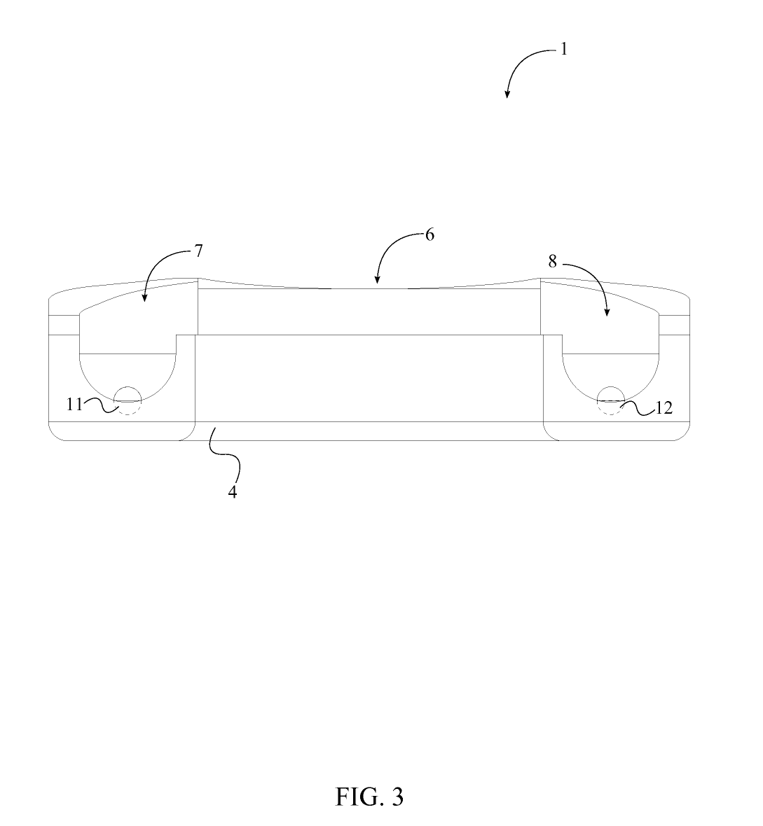

FIG. 4 is a top view of the present invention.

FIG. 5 is a bottom view of the present invention.

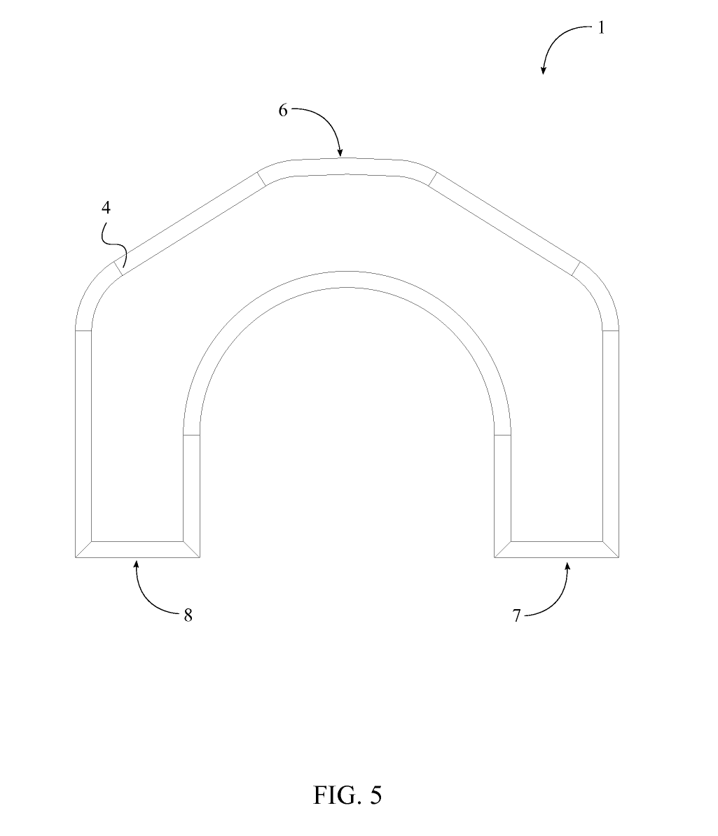

FIG. 6 is a side view of the present invention.

FIG. 7 is another side view of the present invention.

FIG. 8 is a perspective view of one of the plurality of force-sensitive visual indicators.

FIG. 9 is a structural perspective view of the present invention.

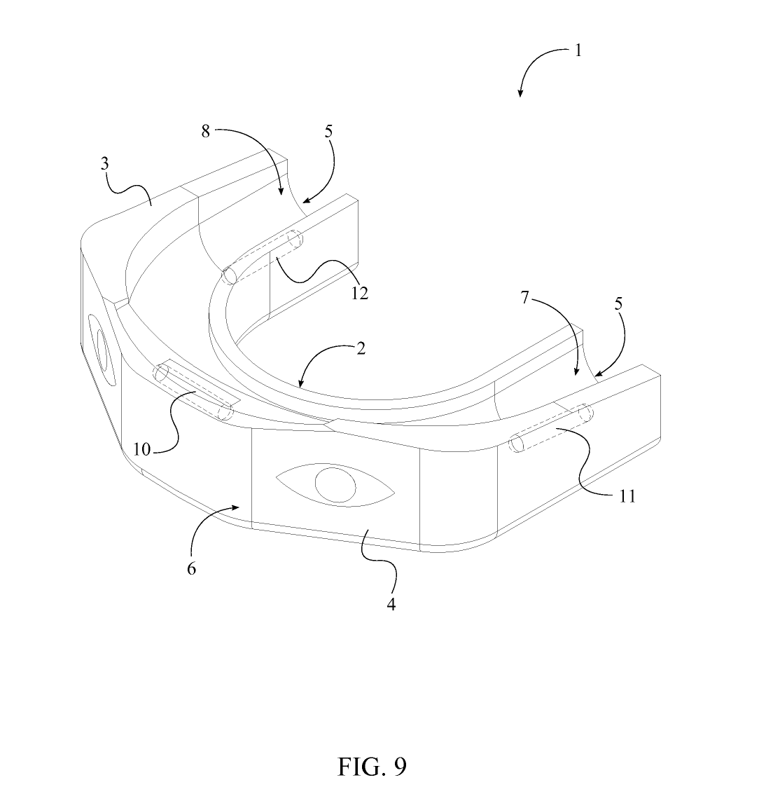

FIG. 10 is a structural top view of the present invention.

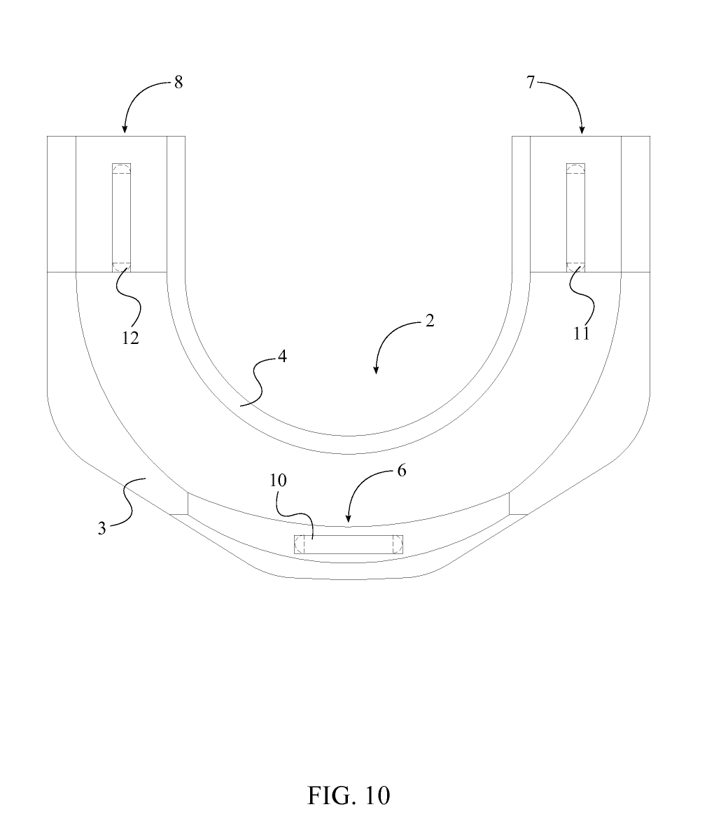

FIG. 11 is a structural rear view of the present invention, wherein the plurality of force-sensitive visual indicators is in the first configuration of the present invention.

FIG. 12 is a cross-sectional view of the first configuration of the present invention.

FIG. 13 is a structural rear view of the present invention, wherein the plurality of force-sensitive visual indicators is in the second configuration of the present invention.

FIG. 14 is a cross-sectional view of the second configuration of the present invention.

FIG. 15 is a top cross-sectional view of one of the plurality of force-sensitive visual indicators.

FIG. 16 is a top view of the present invention, wherein the present invention utilizes a plurality of pressure-sensitive resins.

### DETAIL DESCRIPTIONS OF THE INVENTION

All illustrations of the drawings are for the purpose of describing selected versions of the present invention and are not intended to limit the scope of the present invention.

The present invention is a protective device to be worn by athletes involved in contact sports. More specifically, the present invention is a mouth guard, which is intended to be worn as a precautionary measure, to prevent concussions. The effective design of the present invention indicates the athlete or an observer if the athlete has experienced a significant impact which may lead to concussion.

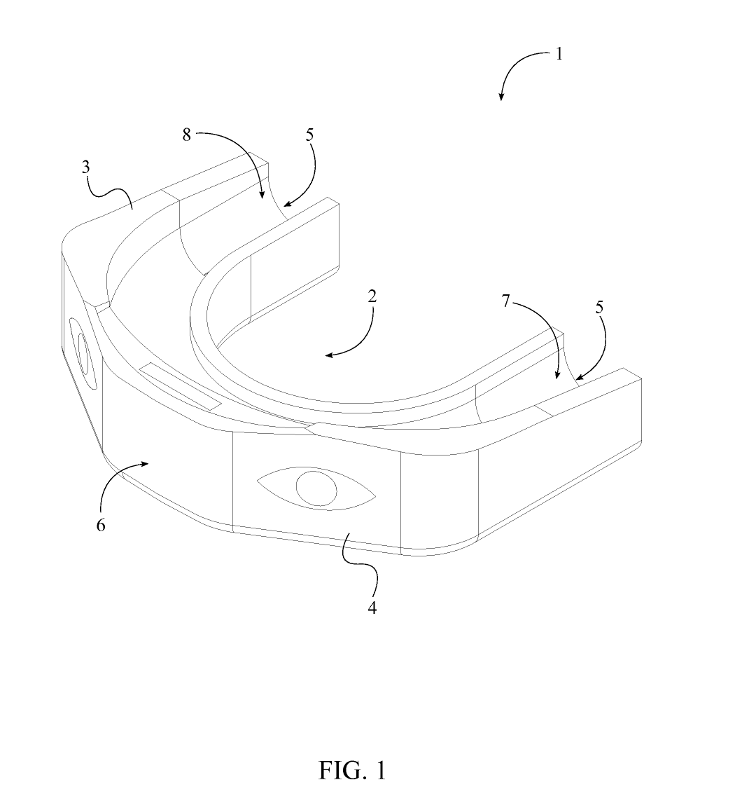

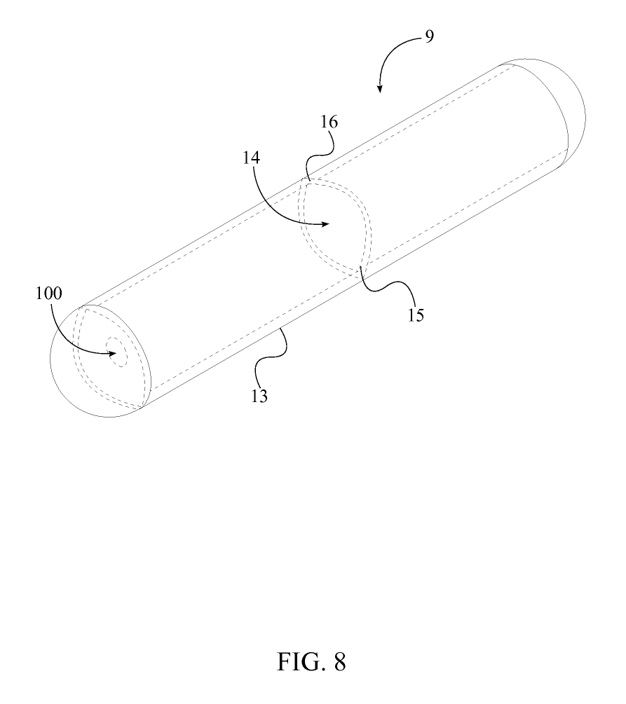

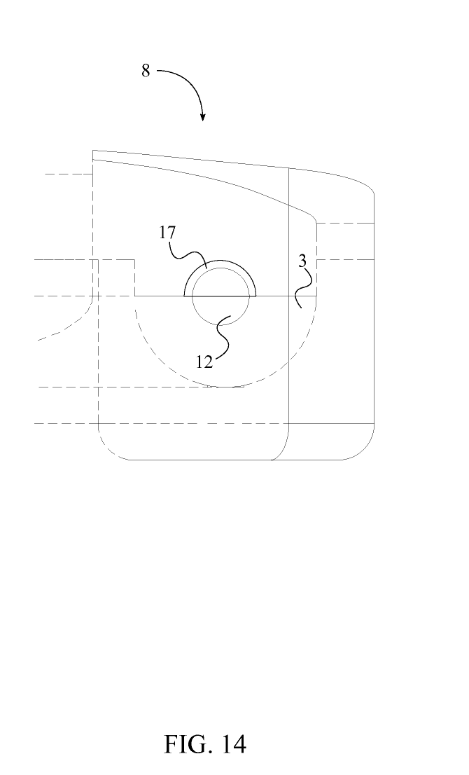

As seen in FIG. 1, FIG. 8, and FIG. 14, the present invention comprises a mouth guard 1, a plurality of force-sensitive visual indicators 9, and a plurality of transparent covers 17. The mouth guard 1 is worn by an athlete when participating in a contact sport. Since the present invention has not deviated from the basic structure of a traditional mouth guard, the athlete can utilize the present invention conveniently. The plurality of force-sensitive visual indicators 9 is utilized to visually indicate an observer of the impact that was experienced by the athlete utilizing the present invention. The observer can get a visual indication of the extent of the impact by viewing the plurality of force-sensitive visual indicators 9 through the plurality of transparent covers 17. In the preferred embodiment of the present invention a color change is used as the visual indication.

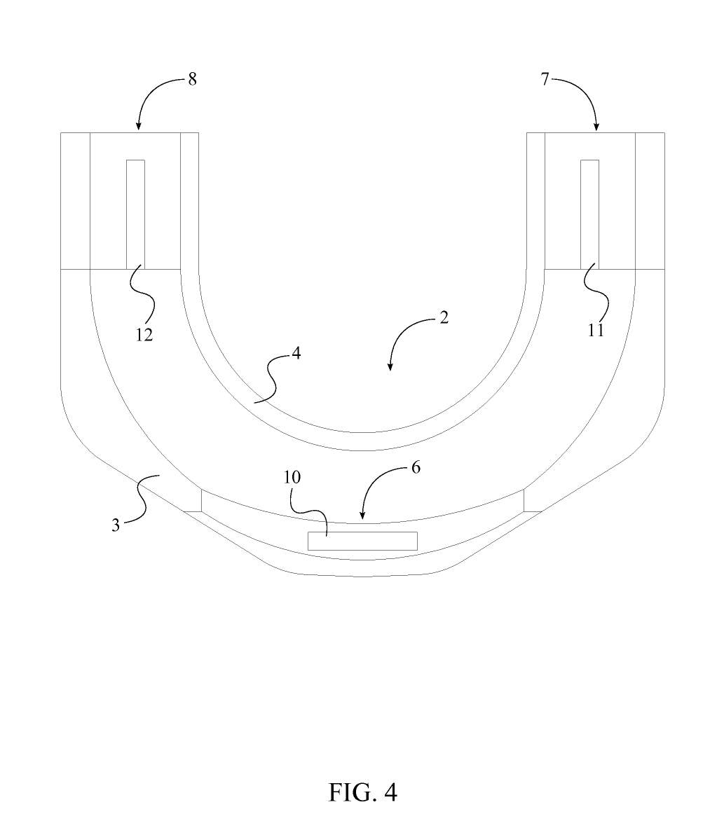

As seen in FIG. 1 and FIG. 4, the mouth guard 1 comprises a U-shaped body 2 and a teeth-receiving channel 5. The U-shaped body 2 is designed to fit the contour of the upper set of teeth comfortably. The teeth-receiving channel 5 receives the upper set of teeth ensuring that the mouth guard 1 is firmly pressed against the upper set of teeth of the user. A firm and comfortable connection with the mouth guard 1 is essential for the user in order to maximize the functionality of the mouth guard 1. In order to provide a comfortable fit, the teeth-receiving channel 5 traverses into the U-shaped body 2. Furthermore, the teeth-receiving channel 5 is positioned along the U-shaped body 2 such that all teeth of the upper set of teeth can be placed within the teeth-receiving channel 5. As a result, each tooth of the upper set of teeth is similarly protected by the present invention. Since the structure of the upper set of teeth, and the general structure of the mouth varies from one user to the other, the present invention is available in a variety of sizes. The varied sizes of the U-shaped body 2 and the teeth-receiving channel 5 ensure that users of all ages can utilize the present invention. As a result, both a child as well as an adult can utilize the present invention with the same effectiveness.

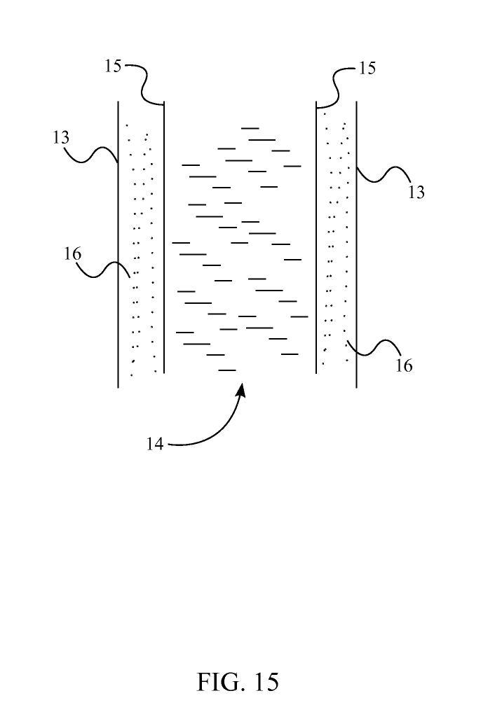

As mentioned earlier, the plurality of force-sensitive visual indicators 9 is utilized to provide visual indication of the dangerousness of the impact. In the preferred embodiment of the present invention, the visual indication is provided by a change of color in each of the plurality of force-sensitive visual indicators 9. In order to do so, each of the plurality of force-sensitive visual indicators 9 comprises an external tubular enclosure 13, a quantity of dye 14, an opaque tubular enclosure 15, and a dye-absorbing material 16. A top cross-sectional view of one of the plurality of force-sensitive visual indicators 9 is illustrated in FIG. 15. Each of the plurality of force-sensitive impact indicators 9 is designed such that the dye-absorbing material 16 absorbs the quantity of dye 14 in order to provide visual indication. In order to do so, the quantity of dye 14 is in fluid communication with the dye-absorbing material 16 through an open-end 100 of the opaque tubular enclosure 15 as illustrated in FIG. 8. The fluid communication between the quantity of dye 14 and the dye-absorbing material 16 allows the dye-absorbing material 16 to provide the visual indication efficiently. The positioning of the opaque tubular enclosure 15 and the dye-absorbing material 16 within the external tubular enclosure 13 is also beneficial in the process of providing an efficient visual indication of a possible concussion causing impact. More specifically, the dye-absorbing material 16 is positioned in between the external tubular enclosure 13 and the opaque tubular enclosure 15 such that the dye-absorbing material 16 can be triggered to provide visual indication within a short time period. In the process of maintaining fluid communication with the dye-absorbing material 16, the quantity of dye 14 is held within the opaque tubular enclosure 15. A liquid surface tension between the quantity of dye 14 and the opaque tubular enclosure 15 assists in retaining the quantity of dye 14 within the opaque tubular enclosure 15. When the athlete who is utilizing the present invention experiences a significant impact, the quantity of dye 14 dissipates into the dye-absorbing material 16. As a result, the dye-absorbing material 16 absorbs the color of the quantity of dye 14 such that a color change is clearly evident. More specifically, the surface tension between the quantity of dye 14 and the opaque tubular enclosure 15 is disturbed, resulting in the quantity of dye 14 dissipating into the dye-absorbing material 16. Since the dye-absorbing material 16 absorbs the quantity of dye 14 that dissipated, the quantity of dye 14 which was previously unseen due to the opaque tubular enclosure 15 is now visible through the external tubular enclosure 13 and the plurality of transparent covers 17. More specifically, the color change in each of the plurality of force-sensitive visual indicators 9 notifies that the athlete has experienced a severe impact. Since the plurality of force-sensitive visual indicators 9 are designed to notify the user at a force below the concussion causing force, the athlete can use the present invention as a precautionary measure in treating the athlete for concussions. In particular, the present invention notifies the athlete that participating in a sport soon after a significant impact can be potentially dangerous.

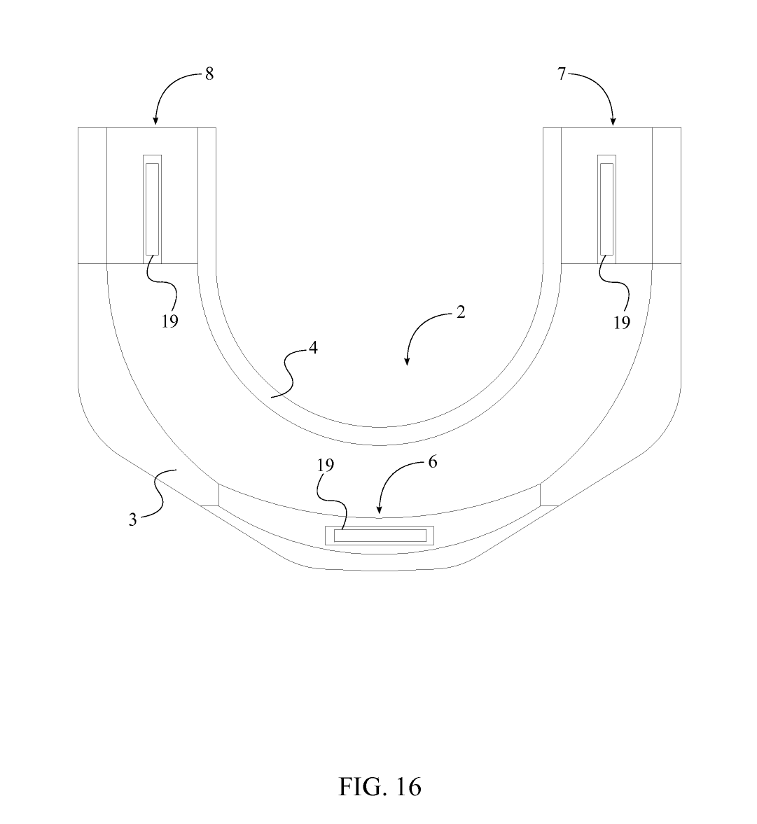

In another embodiment of the present invention, a plurality of pressure-sensitive resins 19 is utilized as the plurality of force-sensitive visual indicators 9 as shown in FIG. 16. Similar to the plurality of force-sensitive visual indicators 9 utilized in the preferred embodiment of the present invention, each of the plurality of pressure-sensitive indicators 19 in the alternative embodiment, also changes color upon experiencing a significant impact. As an example, each of the plurality of pressure-sensitive resins 19 changes from clear to red upon a significant impact.

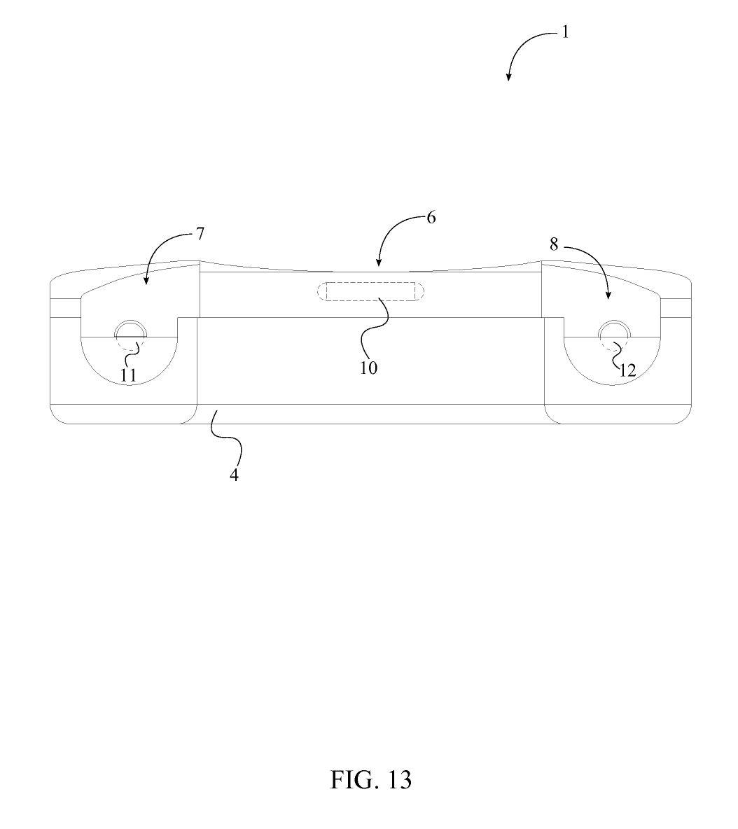

In order to utilize the plurality of force-sensitive visual indicators 9 in conjunction with the mouth guard 1, the plurality of force-sensitive visual indicators 9 is embedded into the U-shaped body 2 from the teeth-receiving channel 5. The direct contact between the mouth guard 1 and the plurality of force-sensitive visual indicators 9 help in efficiently notifying the user of the severity of the impact. In order to provide direct contact, each of the plurality of force-sensitive visual indicators 9 is being enclosed within the U-shaped body 2. In order to provide clear visual indication, each of the plurality of force-sensitive visual indicators 9 is enclosed with a corresponding cover from the plurality of transparent covers 17 as illustrated in FIG. 13 and FIG. 14. More specifically, the corresponding cover is perimetrically connected to the U-shaped body 2 such that the corresponding force-sensitive visual indicator of the plurality of force-sensitive visual indicators 9 can be viewed in its entirety.

As illustrated in FIG. 4, the plurality of force-sensitive visual indicators 9 can be located in multiple functional positions. In order to detect impacts from a wide range of angles, the plurality of force-sensitive visual indicators 9 comprises a front indicator 10. The teeth-receiving channel 5 comprises an incisor section 6 which is used to position the front indicator 10. More specifically, the front indicator 10 is positioned adjacent to the incisor section 6 such that frontal impacts can be accurately identified.

The present invention is designed to detect side-on impacts as well. In order to accurately detect side-on impacts the plurality of force-sensitive visual indicators 9 comprises a left indicator 11 and a right indicator 12. The left indicator 11 and the right indicator 12 are also illustrated in FIG. 4. In order to position the left indicator 11, the teeth-receiving channel 5 comprises a left molar section 7. Likewise, the teeth receiving channel comprises a right-molar section 8 in order to position the right indicator 12. More specifically, the left indicator 11 is positioned adjacent to the left molar section 7. Similarly, the right indicator 12 is positioned adjacent to the right molar section 8. FIG. 4 illustrates the front indicator 10, the left indicator 11, and the right indicator 12 being positioned within the incisor section 6, the left molar section 7, and the right molar section 8 respectively. Even though three visual indicators of the plurality of force-sensitive visual indicators 9 have been utilized in the preferred embodiment of the present invention, a different number of indicators can also be used in order to receive the desired results.

In the alternative embodiment of the present invention, when the plurality of pressure-sensitive resins 19 is utilized, the plurality of pressure-sensitive resins 19 is also positioned adjacent to the incisor section 6, the left molar section 7, and the right molar section 8. Therefore, the plurality of pressure-sensitive resins 19 can detect impacts that occur from multiple directions.

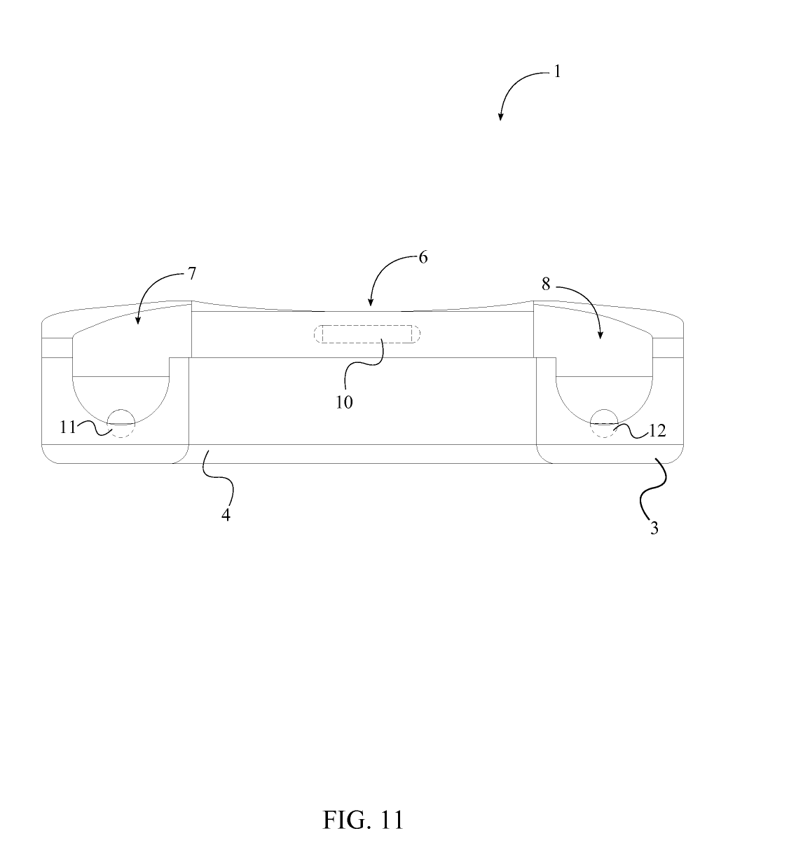

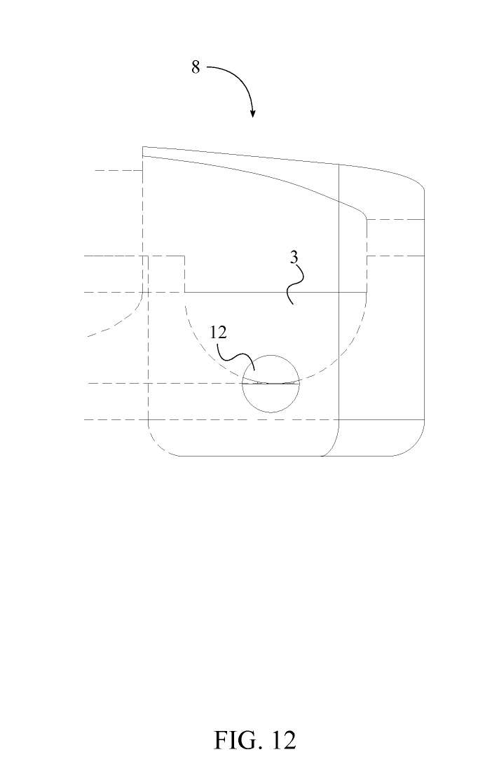

As experienced by many athletes, a sturdy mouth guard 1 can cause discomfort when worn for a long period of time. In order to address this issue, the U-shaped body 2 comprises an external moldable layer 3 and a structural frame 4 which helps in preventing any discomfort to the wearer. The external moldable layer 3 is superimposed onto the structural frame 4. As a result, the teeth of the wearer directly contact the external moldable layer 3 providing a comfortable fit. In a first configuration of the present invention, the plurality of force-sensitive visual indicators 9 is pressed against the structural frame 4 as seen in FIG. 11 and FIG. 12. The firm connection between the external moldable layer 3 and the structural frame 4, allows the plurality of force-sensitive visual indicators 9 to accurately display information. As mentioned earlier, visual indication regarding the force of the impact is received by observing the plurality of force-sensitive visual indicators 9 through the plurality of transparent covers 17. Therefore, in the first configuration of the present invention, the plurality of transparent covers 17 is integrated as a part of the external moldable layer 3.

In a second configuration of the present invention, the external moldable layer 3 is similarly superimposed onto the structural frame 4. However, the plurality of force-sensitive visual indicators 9 is embedded into the external moldable layer 3 in contrast to being pressed against the structural frame 4 as in the first configuration. The plurality of force-sensitive visual indicators 9 being embedded into the external moldable layer 3 is illustrated in FIG. 13 and FIG. 14. Furthermore, FIG. 14 specifically illustrates the right indicator 12 being embedded into the external moldable layer 3. Each of the plurality of force-sensitive visual indicators 9 is enclosed within the external moldable layer 3 and the corresponding cover from the plurality of transparent covers 17. More specifically, the corresponding cover is perimetrically connected to the external moldable layer 3 as illustrated in FIG. 13 and FIG. 14. Therefore, the observer can still detect a color change in the plurality of force-sensitive visual indicators 9 through the corresponding cover.

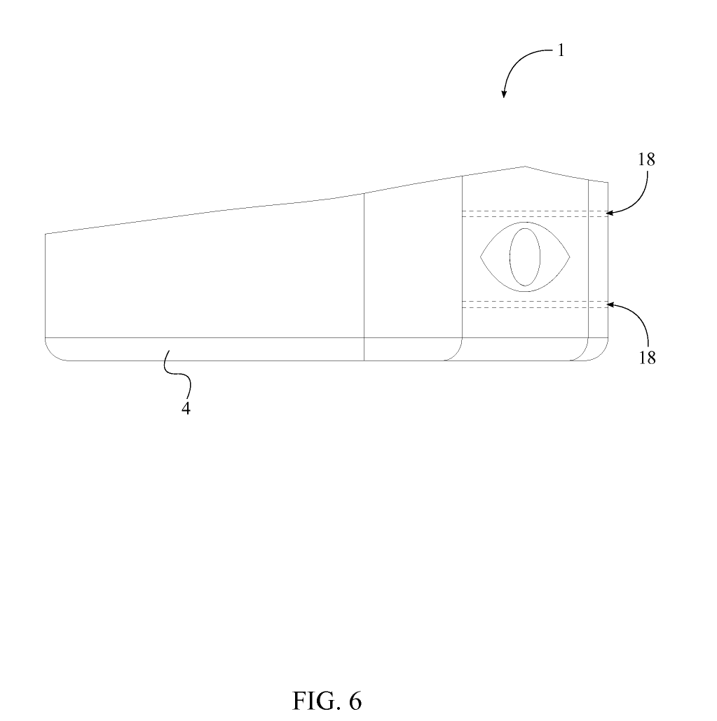

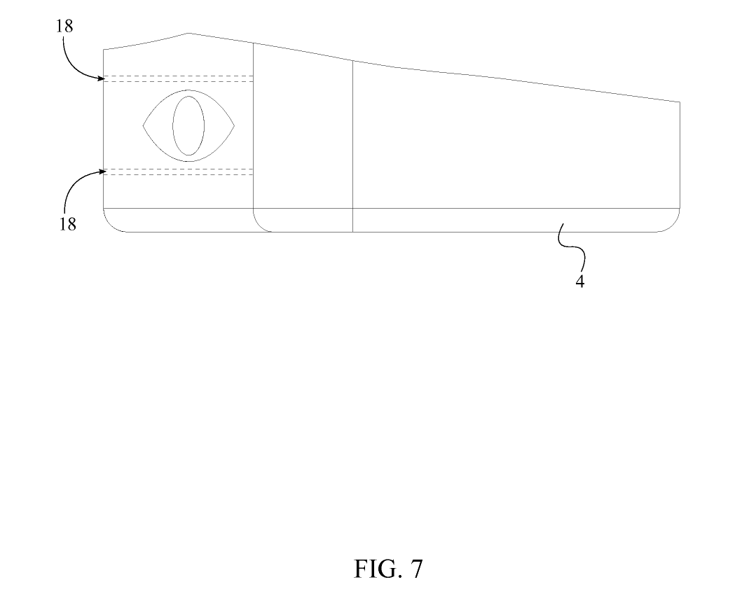

As experienced by athletes on many occasions, wearing a mouth guard can be inconvenient when breathing heavily. The difficulty in breathing while wearing a mouth guard can be a major disadvantage in certain sports such as boxing, karate, and jiu-jitsu. In order to resolve the issue, the present invention comprises a plurality of air passageways 18. The plurality of air passageways 18 traverses through the U-shaped body 2 adjacent to the incisor section 6 in the preferred embodiment of the present invention. The plurality of air passageways 18 is illustrated in FIG. 6 and FIG. 7. However, the plurality of air passageways 18 can be positioned differently with the same effectiveness in another embodiment of the present invention. The plurality of air passageways 18 reduces the need to remove the mouth guard 1 each time the athlete breathes heavily.

The present invention is utilized in the following manner. The mouth guard 1 is worn similar to wearing a traditional mouth guard. The U-shaped body 2 and the teeth-receiving channel 5 help the user wear the present invention comfortably. Additionally, the external moldable layer 3 provides an extra level of comfort to the user. Upon experiencing a significant impact, the mouth guard 1 is removed and each of the plurality of force-sensitive visual indicators 9 is examined through the plurality of transparent covers 17. If the plurality of the force-sensitive visual indicators 9 has changed color from its original color, it is understood that the experienced impact may lead to a concussion. Since the plurality of force-sensitive visual indicators 9 is designed to notify the risk of a concussion at a force below the recognized concussion causing force, the present invention can be utilized as a precautionary measure. However, the present invention does not guarantee to prevent concussions.

Although the invention has been explained in relation to its preferred embodiment, it is to be understood that many other possible modifications and variations can be made without departing from the spirit and scope of the invention as hereinafter claimed.

What is claimed is:

1. An apparatus used to detect concussions in athletes comprises:
a mouth guard;
a plurality of force-sensitive visual indicators;
a plurality of transparent covers;
the mouth guard comprises a U-shaped body and a teeth-receiving channel;
the teeth-receiving channel traversing into the U-shaped body;
the teeth-receiving channel being positioned along the U-shaped body;
the plurality of force-sensitive visual indicators being embedded into the U-shaped body from the teeth-receiving channel;
each of the plurality of force-sensitive visual indicators being enclosed within the U-shaped body and a corresponding cover from the plurality of transparent covers; and
the corresponding cover being perimetrically connected to the U-shaped body.

2. The apparatus used to detect concussions in athletes as claimed in claim 1 comprises:
the teeth-receiving channel comprises an incisor section;
the plurality of force-sensitive visual indicators comprises a front indicator; and
the front indicator being positioned adjacent to the incisor section.

3. The apparatus used to detect concussions in athletes as claimed in claim 1 comprises:
the teeth-receiving channel comprises a left molar section;
the plurality of force-sensitive visual indicators comprises a left indicator; and
the left indicator being positioned adjacent to the left molar section.

4. The apparatus used to detect concussions in athletes as claimed in claim 1 comprises:
the teeth-receiving channel comprises a right molar section;
the plurality of force-sensitive visual indicators comprises a right indicator; and
the right indicator being positioned adjacent to the right molar section.

5. The apparatus used to detect concussions in athletes as claimed in claim 1 comprises:
each of the plurality of the force-sensitive visual indicators comprises an external tubular enclosure, a quantity of dye, an opaque tubular enclosure, and a dye-absorbing material;
the opaque tubular enclosure and the dye-absorbing material being positioned within the external tubular enclosure;
the dye-absorbing material being positioned in between the external tubular enclosure and the opaque tubular enclosure;
the quantity of dye being held within the opaque tubular enclosure by a liquid surface tension between the quantity of dye and the opaque tubular enclosure; and
the quantity of dye being in fluid communication with the dye-absorbing material through an open-end of the opaque tubular enclosure.

6. The apparatus used to detect concussions in athletes as claimed in claim 1 comprises:
the U-shaped body comprises an external moldable layer and a structural frame;
the external moldable layer being superimposed onto the structural frame;
the plurality of force-sensitive visual indicators being pressed against the structural frame; and
the plurality of transparent covers being integrated as a part of the external moldable layer.

7. The apparatus used to detect concussions in athletes as claimed in claim 1 comprises:
the U-shaped body comprises an transparent moldable layer and an structural frame;
the external moldable layer being superimposed onto the structural frame;
the plurality of force-sensitive visual indicators being embedded into the external moldable layer;
each of the plurality of force-sensitive visual indicators being enclosed within the external moldable layer and the corresponding cover from the plurality of transparent covers; and
the corresponding cover being perimetrically connected to the external moldable layer.

8. The apparatus used to detect concussions in athletes as claimed in claim 1 comprises:
a plurality of air passageways;
the teeth-receiving channel comprises an incisor section; and
the plurality of air passageways traversing through the U-shaped body adjacent to the incisor section.

9. The apparatus used to detect concussions in athletes as claimed in claim 1, wherein each of the plurality of the force-sensitive visual indicators is a pressure-sensitive resin.

10. An apparatus used to detect concussions in athletes comprises:
a mouth guard;
a plurality of force-sensitive visual indicators;
a plurality of transparent covers;
the mouth guard comprises a U-shaped body and a teeth-receiving channel;
the teeth-receiving channel traversing into the U-shaped body;
the teeth-receiving channel being positioned along the U-shaped body;
the plurality of force-sensitive visual indicators being embedded into the U-shaped body from the teeth-receiving channel;
each of the plurality of force-sensitive visual indicators being enclosed within the U-shaped body and a corresponding cover from the plurality of transparent covers; and
the corresponding cover being perimetrically connected to the U-shaped body,
the teeth-receiving channel comprises an incisor section;
the plurality of force-sensitive visual indicators comprises a front indicator; and
the front indicator being positioned adjacent to the incisor section. the teeth-receiving channel comprises a left molar section;
the plurality of force-sensitive visual indicators comprises a left indicator;
the left indicator being positioned adjacent to the left molar section. the teeth-receiving channel comprises a right molar section;
the plurality of force-sensitive visual indicators comprises a right indicator; and
the right indicator being positioned adjacent to the right molar section.

11. The apparatus used to detect concussions in athletes as claimed in claim 10 comprises:
each of the plurality of the force-sensitive visual indicators comprises an external tubular enclosure, a quantity of dye, an opaque tubular enclosure, and a dye-absorbing material;
the opaque tubular enclosure and the dye-absorbing material being positioned within the external tubular enclosure;
the dye-absorbing material being positioned in between the external tubular enclosure and the opaque tubular enclosure;
the quantity of dye being held within the opaque tubular enclosure by a liquid surface tension between the quantity of dye and the opaque tubular enclosure; and
the quantity of dye being in fluid communication with the dye-absorbing material through an open-end of the opaque tubular enclosure.

12. The apparatus used to detect concussions in athletes as claimed in claim 10 comprises:
the U-shaped body comprises an external moldable layer and a structural frame;
the external moldable layer being superimposed onto the structural frame;
the plurality of force-sensitive visual indicators being pressed against the structural frame; and
the plurality of transparent covers being integrated as a part of the external moldable layer.

13. The apparatus used to detect concussions in athletes as claimed in claim 10 comprises:
the U-shaped body comprises an transparent moldable layer and an structural frame;
the external moldable layer being superimposed onto the structural frame;
the plurality of force-sensitive visual indicators being embedded into the external moldable layer;
each of the plurality of force-sensitive visual indicators being enclosed within the external moldable layer and the corresponding cover from the plurality of transparent covers; and
the corresponding cover being perimetrically connected to the external moldable layer.

14. The apparatus used to detect concussions in athletes as claimed in claim 10 comprises:
a plurality of air passageways;
the teeth-receiving channel comprises an incisor section; and
the plurality of air passageways traversing through the U-shaped body adjacent to the incisor section.

15. The apparatus used to detect concussions in athletes as claimed in claim 10, wherein each of the plurality of the force-sensitive visual indicators is a pressure-sensitive resin.
